SEQUENCE LISTING (6 sequences)

SEQ ID NO: 1 - DNA, 25 bp, Artificial Sequence
  Synthetic LAIV-A forward primer
gaagcaaagattgaacaagagaagc

SEQ ID NO: 2 - DNA, 26 bp, Artificial Sequence
  Synthetic LAIV-A reverse primer
cacaaatacttctcgctagtgtttcg

SEQ ID NO: 3 - DNA, 31 bp, Artificial Sequence
  Syntehtic LAIV-A probe
cactgacattgaacacaatgactaaagatgc

SEQ ID NO: 4 - DNA, 18 bp, Artificial Sequence
  Synthetic LAIV-B forward primer
atgagttggggctggcta

SEQ ID NO: 5 - DNA, 20 bp, Artificial Sequence
  Syntehtic LAIV-B reverse primer
ccacagcttccataagaagt

SEQ ID NO: 6 - DNA, 19 bp, Artificial Sequence
  Synthetic LAIV-B Probe
ttcttggacttcccttcag

(19) United States
(12) Patent Application Publication
(10) Pub. No.: US 2018/0371527 A1
(43) Pub. Date: Dec. 27, 2018

(54) DETECTION OF LIVE ATTENUATED INFLUENZA VACCINE VIRUSES

(71) Applicant: THE UNITED STATED OF AMERICA AS REPRESENTED BY THE SECRETARY OF THE DEPARTMENT OF HEALTH AND HUMAN S, Rockville, MD (US)

(72) Inventors: Bo SHU, Atlanta, GA (US); Stephen LINDSTROM, Atlanta, GA (US); Kai-Hui WU, Atlanta, GA (US); LaShondra BERMAN, Atlanta, GA (US); Christine WARNES, Atlanta, GA (US)

(73) Assignee: The United States of America as Represented by the Secretary of the Department of Health and Human S, Rockville, MD (US)

(21) Appl. No.: 16/062,483

(22) PCT Filed: Dec. 13, 2016
(86) PCT No.: PCT/US2016/066370

§ 371 Date: Jun. 14, 2018

Related U.S. Application Data
(60) Provisional application No. 62/267,085, filed on Dec. 14, 2015.

(51) Int. Cl. C12Q 1/686; C12Q 1/6806; C12Q 1/70; C12Q 1/6853

(52) U.S. Cl. CPC C12Q 1/686; C12Q 1/6806; C12Q 1/701; C12Q 2600/166; C12Q 2600/112; C12Q 2600/16; C12Q 1/6853

(57) ABSTRACT

Primers, probes and kits for detection of live attenuated influenza vaccine (LAIV) virus strains are provided. Also provided are the assays for detecting LAIV.

### PRIOR RELATED APPLICATION

The present application claims the benefit of priority of U.S. Provisional Application No. 62/267,085 filed Dec. 14, 2015, the content of which is incorporated herein by reference in its entirety.

### FIELD

The invention is related to compositions and method for the detection of influenza virus.

### BACKGROUND

Administration of live attenuated influenza vaccines, such as FluMist® (Medimmune, Gaithersburg, Md.), is an effective and cost-effective preventive measure against influenza infections. Currently available live attenuated influenza vaccines are formulated for intranasal administration and continue to replicate in nasopharynx for several days or even weeks after administration. Occasionally, live attenuated influenza vaccines can cause influenza, particularly in people with a weakened immune system. More commonly, live attenuated influenza vaccines cause transient respiratory symptoms in the inoculated individuals. The samples obtained from the inoculated individuals may be falsely positive for one or multiple circulating influenza virus targets in clinical diagnostic tests due to cross-reactivity. For both clinical and epidemiological reasons, is important to have sensitive and specific diagnostics tests that can distinguish between the strains of influenza found in live attenuated influenza vaccines and the seasonal circulating influenza virus strains.

### SUMMARY

As described below, the inventors have discovered PCR primers and probes that are useful for detecting Live Attenuated Influenza Vaccine (LAIV) virus strains with high specificity and sensitivity by PCR-based assays. The primer and the probes discovered by the inventors can be used in the detection methods that employ reverse transcriptase (RT-PCR) techniques that monitor the amplification of LAIV virus strains in real time. Thus, real-time RT-PCR (rRT-PCR) assays were developed for detection of LAIV viruses strain. The primers and the probes discovered by the inventors can also be combined in kits for conducting such assays. Accordingly, the present invention provides PCR primers, PCR probes, methods of using the PCR primers and/or probes, as well as the kits comprising the probes and/or primers. The embodiments of the present invention can be variously applied in clinical, research and public health filed. For example, embodiments of the present invention can be used to determine if samples of interest, such as those obtained from individuals having influenza-like symptoms contain a LAIV virus strain. This information can be used to determine if an individual is infected with a LAIV virus strain or by community-acquired influenza virus strain. In an additional application, embodiments of the present invention can be used to detect LAIV virus strains in influenza vaccine samples, for example, as a quality control measure.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are further described in the Detailed Description section below.

Some of the embodiments of the present invention are summarized below. This summary is not intended to identify key or essential features of the claimed subject matter, nor is it intended to be used in isolation to determine the scope of the claimed subject matter. The subject matter should be understood by reference to appropriate portions of the entire specification and each claim.

Some embodiments of the present invention are probes, which can be useful for performing real time PCR assays. One example is a probe comprising an oligonucleotide comprising a sequence at least 90% identical to SEQ ID NO:3 or SEQ ID NO:6, linked to at least one of a fluorophore moiety and a fluorescence quencher moiety. The probe can have a length of 35 bases or less. The sequence can be SEQ ID NO:3 or SEQ ID NO:6, or the oligonucleotide can consist of SEQ ID NO:3 or SEQ ID NO:6. The probe can be an oligonucleotide consisting of SEQ ID NO:3 or SEQ ID NO:6 linked to the fluorophore moiety and the fluorescence quencher moiety. In any of the above example, the fluorophore moiety can comprise a fluorescein moiety. The fluorophore moiety can be coupled to a 5′ terminus of the probe. In any of the above examples, the fluorescence quencher moiety can be a dark quencher, such as a BHQ quencher. The fluorescence quencher moiety can be coupled to a 3′ terminus of the probe or to an internal base.

Some embodiments of the present invention are primers, which can be useful for performing PCR amplification, including amplification during real time PCR assay. An example is a primer comprising an oligonucleotide comprising a sequence at least 90% identical to a sequence selected from the group consisting of SEQ ID NO:1, SEQ ID NO:2. SEQ ID NO:4, and SEQ ID NO:5. The above primer can comprise a fluorescent moiety, quencher moiety or both. The primer can have a length of 30 bases or less. In the primers according to the embodiments of the present invention, the sequence can be selected from the group consisting of SEQ ID NO:1, SEQ ID NO:2, SEQ ID NO:4, and SEQ ID NO:5. For example, the oligonucleotide can consist of the sequence selected from the group consisting of SEQ ID NO: 1, SEQ ID NO:2, SEQ ID NO:4, and SEQ ID NO:5.

Some other embodiments of the present invention are kits. One example is a kit for detecting a Live Attenuated Influenza Vaccine (LAIV) virus strain in a sample, comprising at least one probe of those described above and other reagents for performing a real time reverse transcriptase (rRT-PCR) assay. The other reagents can comprise at least one primer comprising a sequence at least 90% identical to a sequence selected from the group consisting of SEQ ID NO:1, SEQ ID NO:2, SEQ ID NO:4, and SEQ ID NO:5. For example, the kit can comprise a probe comprising the sequence at least 90% identical to SEQ ID NO:3 and at least one primer selected from the group consisting of a first primer comprising a sequence at least 90% identical to SEQ ID NO:1 and a second primer comprising a sequence at least 90% identical to SEQ ID NO:2. In another example, the kit can comprise a probe comprising the sequence at least 90% identical to SEQ ID NO:6 and at least one primer selected from the group consisting of a first primer comprising a sequence at least 90% identical to a sequence SEQ ID NO:4 and a second primer comprising a sequence at least 90% identical to a sequence SEQ ID NO:5. Another example of a kit is a kit for amplifying a region of a LAIV virus strain gene in a sample, comprising at least one primer of those described above and one or more other reagents for performing a PCR. One more example is a kit for amplifying a region of a gene of a LAIV virus strain in a sample, comprising at least one of: one or both primers for amplifying a region of PB1 gene of LAIV-A virus selected from the group consisting of a first primer comprising SEQ ID NO: 1 or an oligonucleotide of a sequence at least 90% identical to SEQ ID NO:1 and a second primer comprising SEQ ID NO:2 or an oligonucleotide of a sequence at least 90% identical to SEQ ID NO:2, one or both primers for amplifying a region of PA gene of LAIV-B virus selected from the group consisting of a third primer comprising SEQ ID NO:4 or an oligonucleotide of a sequence at least 90% identical to SEQ ID NO:4 and a fourth primer comprising SEQ ID NO:5 or an oligonucleotide of a sequence at least 90% identical to SEQ ID NO:5, and one or more other reagents for performing a PCR. In the above example, the kit can comprise the primers for amplifying a region of PB1 gene of LAIV-A virus and the primers for amplifying a region of PA gene of LAIV-B virus. The reagents for performing PCR including in a kit can be the reagents for performing RT-PCR, such as rRT-PCR. At least one of the primers included in a kit can comprise a fluorescent moiety, a quencher moiety, or both. The other reagents included in a kit can comprise one or more probe (probes) or additional primer (primers). In one example, the other reagents can comprise at least one of a first probe, comprising an oligonucleotide comprising a sequence at least 90% identical to SEQ ID NO:3, if the one or both primers for amplifying a region of PB1 gene of LAIV-A virus are preset in the kit, and a second probe comprising an oligonucleotide comprising a sequence at least 90% identical SEQ ID NO:6, if one or both primers for amplifying a region of PA gene of LAIV-B virus are present in the kit. The first probe and the second probe comprise at least one of a fluorophore moiety and a fluorescence quencher moiety. The region of PB1 gene of LAIV-A virus can be derived from attenuated cold-adapted influenza virus A/Ann Arbor/06/1960-ca or A/Leningrad/134/1957-ca; the region of PA gene of LAIV-B virus can be derived from attenuated cold-adapted influenza virus B/Ann Arbor/01/1966-ca or B/USSR/60/1969-ca. In any of the above examples, the sample can be an ex vivo sample derived from a human subject, a laboratory sample, a virus isolate sample, or a vaccine sample.

Embodiments of the present invention also include methods. One example is a method of detecting a presence or absence of an influenza strain in a sample, wherein the influenza virus strain comprises a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising the steps of: contacting the sample with reagents for performing rRT-PCR and a probe of any one of the probes described above that are specific for the region of LAIV-A PB1 gene (LAIV-A probe) and forward and reverse primers specific for the region of LAIV-A PB1 gene (LAIV-A primer), or a probe of any one of the probes described above that are specific for the region of LAIV-B PA gene (LAIV-B probe) and forward and reverse primers specific for the region of LAIV-B PA gene (LAIV-B primer), performing rRT-PCR on the sample to generate a PCR cycle threshold; and, comparing the PCR cycle threshold to a control value. In the above method, if the cycle threshold is below the control value, the LAIV influenza strain is absent from the sample, and if the cycle threshold is above the control value, the LAIV influenza strain is present in the sample. The sample can be contacted with LAIV-A probe and forward LAIV-A primer comprising a sequence at least 90% identical to SEQ ID NO:1. The sample can be contacted with LAIV-A probe and a reverse LAIV-A primer comprising a sequence at least 90% identical to SEQ ID NO:2. The sample can be contacted with LAIV-B probe and forward LAIV-B primer comprising a sequence at least 90% identical to SEQ ID NO:4. The sample can be contacted with LAIV-B probe and a reverse LAIV-B primer comprising a sequence at least 90% identical to SEQ ID NO:5. Any of the above examples of the method can further comprise a step of determining a quantity of the LAIV virus strain in the sample when the LAIV virus strain is present in the sample. The steps of comparing, the determining the quantity, or both, can be performed by a computer.

One more example of a method according to the embodiments of the present invention is a method of amplifying a region of a gene of LAIV virus strain in a sample, wherein the region is a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising the steps of: contacting the sample with at least one primer of any one of the primers described above that are specific for the region of LAIV-A PB1 gene (LAIV-A primer) or specific for the region of LAIV-B PA gene (LAIV-B primer); and, performing a PCR. In the above method, the LAIV-A primer can be a forward primer comprising the sequence at least 90% identical to SEQ ID NO: 1 or a reverse primer comprising the sequence at least 90% identical to SEQ ID NO:2. The LAIV-B primer can be a forward primer comprising the sequence at least 90% identical to SEQ ID NO:4 or a reverse primer comprising the sequence at least 90% identical to SEQ ID NO:5. The PCR can be RT-PCR. The above methods can be performed as methods of detecting the LAIV virus strain in the sample, when further comprising a step of detecting one or more products of the amplification, when the LAIV virus strain is present in the sample if the one or more products of the amplification are detected corresponding to the region of LAIV-A PB1 gene or LAIV-B PA gene. In such case, the PCR is rRT-PCR and the detecting of the region of LAIV-A PB1 gene can be performed using a probe of any one of the probes described above that are specific for the region of LAIV-A PB1 gene (LAIV-A probe). The detecting of the region of LAIV-A PB1 gene can be performed using a probe of any one of the probes described above that are specific for the region of LAIV-B PA gene (LAIV-B probe). The above methods can further comprise a step of determining a quantity of the LAIV virus strain when the LAIV virus strain is present in the sample. The sample can be an ex vivo sample derived from a human subject, a laboratory sample, a virus isolate sample, or a vaccine sample.

Yet one more example of a method according to the embodiments of the present invention is a method of determining if a patient is infected with a LAIV virus strain, wherein the LAIV virus strain comprises a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising the steps of: contacting a sample derived from the patient with reagents for performing rRT-PCR and a probe of any one the probes described above that are specific for the region of LAIV-A PB1 gene (LAIV-A probe) and forward and reverse primers specific for the region of LAIV-A PB1 gene (LAIV-A primer), or a probe of any one the probes described above that are specific for the region of LAIV-B PA gene (LAIV-B probe) and forward and reverse primers specific for the region of LAIV-B PB-A gene (LAIV-B primer), performing rRT-PCR on the sample to generate a PCR cycle threshold, and, comparing the PCR cycle threshold to a control value, wherein if the cycle threshold is below the control value, the patient is not infected with the LAIV virus strain, and wherein if the cycle threshold is above the control value, the patient is infected with the LAIV virus strain. In the above method, the sample can be contacted with LAIV-A probe and forward LAIV-A primer comprising a sequence at least 90% identical to SEQ ID NO:1. The sample can be contacted with LAIV-A probe and a reverse LAIV-A primer comprising a sequence at least 90% identical to SEQ ID NO:2. The sample can be contacted with LAIV-B probe and forward LAIV-B primer comprising a sequence at least 90% identical to SEQ ID NO:4. The sample can be contacted with LAIV-B probe and a reverse LAIV-B primer comprising a sequence at least 90% identical to SEQ ID NO:5. Any of the above methods can further comprise a step of determining a quantity of the LAIV virus strain in the sample when the LAIV virus strain is present in the sample. The steps of comparing, the determining the quantity, or both, can be performed by a computer.

One more example is a method of determining if a patient is infected with a LAIV virus strain, wherein the LAIV virus strain comprises a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising the steps of: performing a RT-PCR on a sample derived from the patient using at least one primer of any one of the primers described above; and, detecting one or more products of the PCR, wherein the LAIV virus strain is present in the sample if the one or more products of the amplification are detected corresponding to a gene region derived from LAIV-A or LAIV-B. In the above method, the PCR can be rRT-PCR, the at least one primer can be a forward primer comprising the sequence at least 90% identical to SEQ ID NO:1 or a reverse primer comprising the sequence at least 90% identical to SEQ ID NO:2, and the detecting of the region of LAIV-A PB1 gene can be performed using a probe of any one of the probes described above that are specific for the region of LAIV-A PB1 gene (LAIV-A probe). The at least one primer can be a forward primer comprising the sequence at least 90% identical to SEQ ID NO:4 or a reverse primer comprising the sequence at least 90% identical to SEQ ID NO:5, the PCR can be rRT-PCR, and the detecting of the region of LAIV-A PB1 gene can be performed using a probe of any one of the probes described above that are specific for the region of LAIV-B PA gene (LAIV-B probe).

Other objects and advantages of the invention will be apparent from the following detailed description of embodiments of the invention.

### BRIEF DESCRIPTION OF THE FIGURES

FIGS. 1A, 1B, 1C and 1D show the examples of the chemical structures of Black Hole Quencher® dyes (Biosearch Technologies, Petaluma, Calif.).

FIG. 2 is a schematic illustration of TaqMan probe.

FIG. 3 is a schematic illustration of Zen probe.

FIG. 4 shows chemical structures of pdU-CE Phosphoramidite (5′-Dimethoxytrityl-5-(1-Propynyl)-2′-deoxyUridine, 3′-[(2-cyanoethyl)-(N,N-diisopropyl)]-phosphoramidite) and pdC-CE Phosphoramidite (5′-Dimethoxytrityl-N4-diisobutylaminomethylidene-5-(1-Propynyl)-2′-deoxyCytidine, 3′-[(2-cyanoethyl)-(N,N-diisopropyl)]-phosphoramidite).

### DESCRIPTION

### Definitions

The following abbreviations may be used in the present documents: LAIV—live attenuated influenza vaccine; PCR—polymerase chain reaction; RT—reverse transcriptase; RT-PCR—reverse transcriptase PCR; rRT-PCR—real-time RT-PCR RNA—ribonucleic acid; DNA—deoxyribonucleic acid; PB1—polymerase basic 1; PA—polymerase acidic; HA—hemaglutinin; NA—neuramidase; BHQ-Black Hole Quencher, FAM—6 carboxyfluorescein; FRET—fluorescence resonance energy transfer; TET—tetrachlorofluorescein, hexacholoro-6-carboxyfluorescein (HEX).

The term “amplification” and the related terms are used to refer to the process or to the result of the process used to increase the number of copies of a nucleic acid molecule. The resulting products can be called “amplification products” or “amplicons.” An example of amplification technique is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

The term “assay” and the related terms are used to broadly refer to methods, processes or procedures used for assessing or measuring the presence, absence or amount or the of a target entity (the analyte). The assays according to the embodiments of the present invention are used to describe the presence, absence or amount of LAIV virus strain in a sample.

The terms “assess.” “assessment.” “assessing” and the related terms are used in reference to influenza virus and its genes to denote inferring the presence or the absence of influenza virus strain in a sample based on the detected presence or absence of the detected regions of influenza virus genes.

The terms “to contact,” “contacting” and the related terms can be used to describe the process or the result of placing chemical compounds in the same reaction environment, such as the same reaction vessel or solution.

The terms “detect,” “detecting,” “detection” and similar terms are used in this document to broadly to refer to a process or discovering or determining the presence or an absence, as well as a degree, quantity, or level, or probability of occurrence of something. The terms necessarily involve a physical transformation of matter, such as nucleic acid amplification by PCR. For example, the term “detecting” when used in the context of influenza virus strain detection, can denote discovery or determination of the presence, absence, level or quantity, as well as a probability or likelihood of the presence or absence of the influenza virus strain being detected. It is to be understood that the expressions “detecting presence or absence,” “detection of presence or absence” and related expressions, include qualitative, semi-quantitative and quantitative detection. Quantitative detection includes the determination of level, quantity or amounts of influenza virus in the sample, on which the detection process is performed. Semi-quantitative detection and qualitative detection include inferring the presence or absence of a strain of influenza virus in a sample based on a detection parameter being above or below a predetermined value.

The terms “detection limit.” “limit of detection” and other related terms can be used in the context of the embodiments of the present invention to refer to the lowest analyte concentration that can be reliably (for example, reproducibly) detected for a given type of sample and/or assay. Limit of detection can be determined by testing serial dilutions of a sample known to contain the analyte and determining the lowest dilution at which detection occurs. The limit of detection of the assays described in this document can be expressed as level of infectivity (for example, 50% tissue culture infective dose/ml (TCID₅₀/ml) or 50% embryo (or egg) infective dose/ml (EID₅₀/ml), expressed as a log scale) or RNA copy number/μl that can be detected.

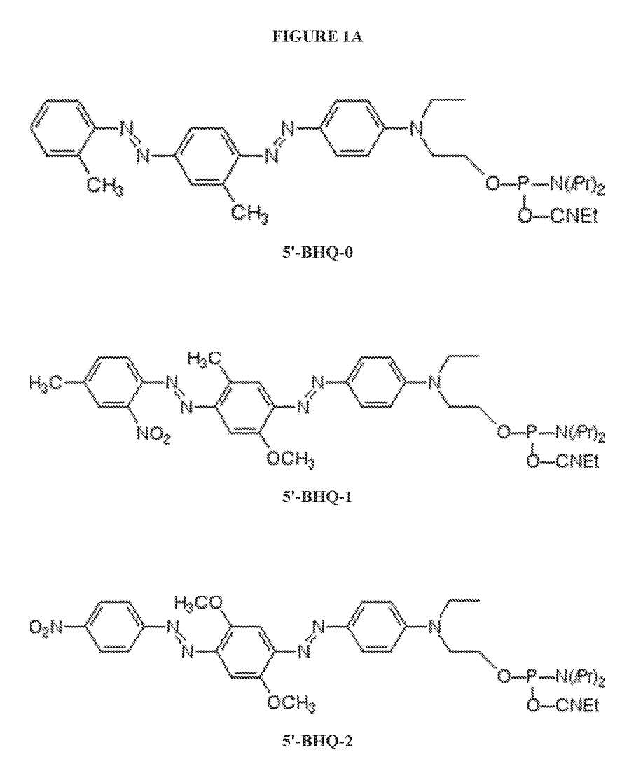

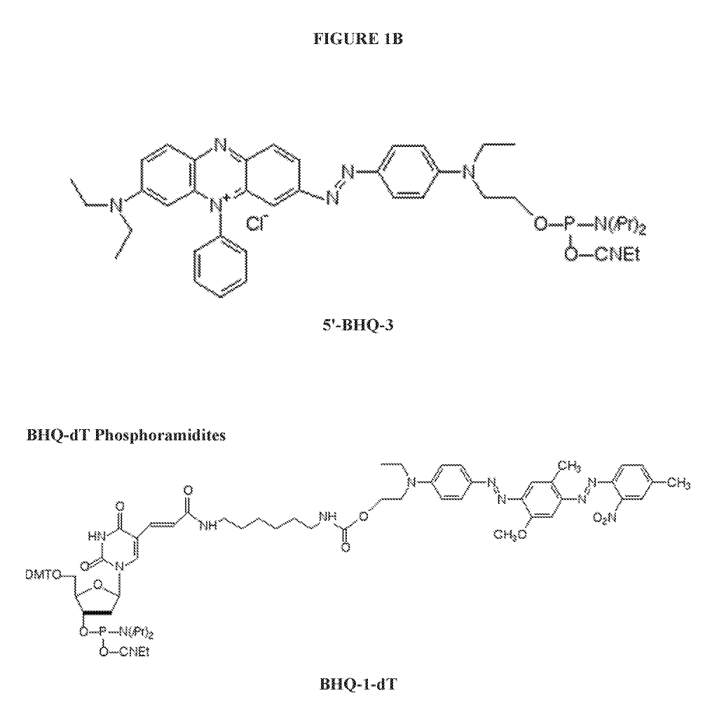

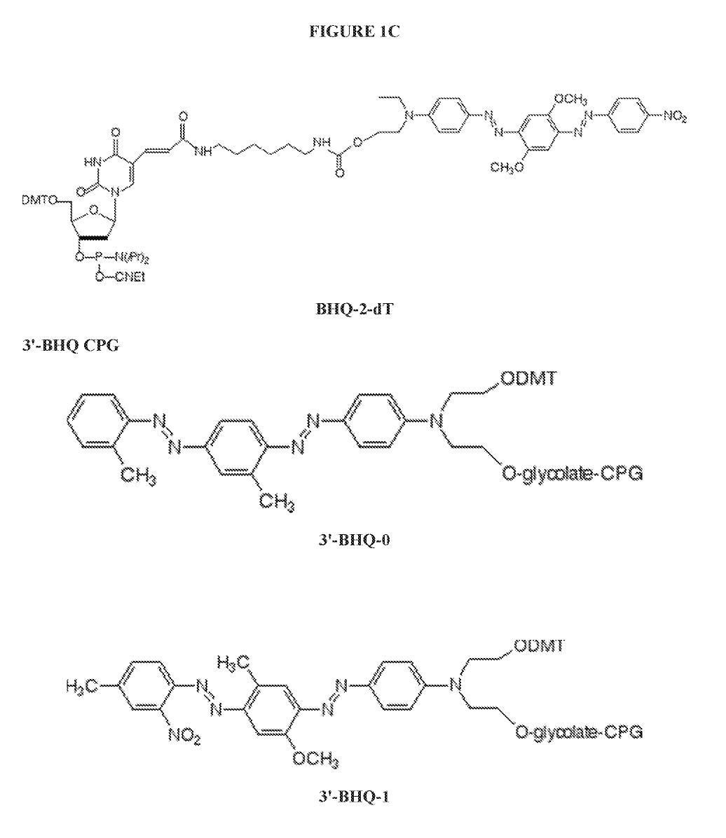

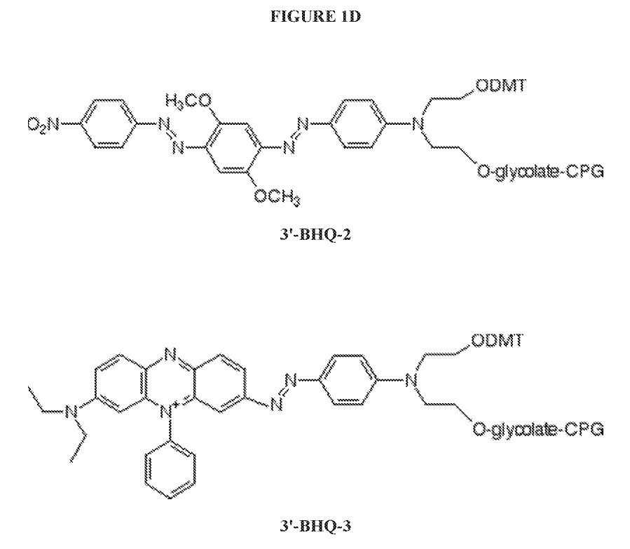

The term “fluorescence” broadly refers to the process or the result of the emission of light by a substance that has absorbed light or other electromagnetic radiation. The following terms and concepts can be used to describe how fluorescence is employed in the embodiments of the present invention. Fluorophores or fluorescent dyes are chemical compounds or moieties that can re-emit light upon light excitation. Fluorophores typically contain several combined aromatic groups, or plane or cyclic molecules with several n bonds. A fluorophore absorbs light energy of a specific wavelength and re-emits light at a longer wavelength. When a fluorophore is excited at a particular wavelength, it is promoted to an excited state. In the absence of a quencher, the excited dye emits light in returning to the ground state. When a quencher is present in physical proximity, the excited fluorophore can return to the ground state by transferring its energy to the quencher, without the emission of light. Different types of quenchers exist. One quenching mechanism relies on the ability of the fluorophore to transfer energy to a second fluorophore by fluorescence resonance energy transfer (FRET). This returns the fluorophore to the ground state and generates the quencher excited state. The quencher then returns to the ground state through emissive decay (fluorescence). In order for this to happen, the emission spectrum of the fluorophore must overlap with the absorption spectrum of the second fluorophore (quencher). One example of such the fluorophore/quencher pair is fluorescein (used as the fluorescent reporter dye) and rhodamine as the quencher (FAM/TAM probes). However, quencher fluorescence can increase background noise due to overlap between the quencher and reporter fluorescence spectra. Dark quenchers are dyes with no native fluorescence. Dark quenchers return from the excited state to the ground state via non-radiative decay pathways, without the emission of light. In dark decay, energy is given off via molecular vibrations (heat). With the typical μM or less concentration of probe, the heat from radiationless decay is too small to affect the temperature of the solution. Thus, the term “dark quencher” can be used in the context of the present invention to refer to a substance or moiety that absorbs excitation energy from a fluorophore and dissipates the energy as heat; while the term “fluorescent quencher” can be used to refer to a substance or moiety that re-emits much of this energy as light. Dark quenchers do not occupy an emission bandwidth and allow multiplexing, when two or more reporter-quencher probes are used together. BHQ quenchers, some of which are illustrated of FIG. 1, are examples of dark quenchers.

Influenza (flu) virus is a member of Orthomyroviridae family. There are three subtypes of influenza viruses, designated influenza A, influenza B, and influenza C. Human influenza A and B viruses cause seasonal epidemics of disease almost every winter in the United States. The emergence of a novel and different influenza virus strain infecting people can cause an influenza pandemic. Influenza type C infections cause a mild respiratory illness and are not thought to cause epidemics. Influenza virus is an RNA virus and contains a segmented negative-sense RNA genome. That is, influenza type virus genome is not a single piece of RNA; instead, it consists of segmented pieces of negative-sense RNA, which can be referred to as “segments,” each piece containing either one or two genes which code for a gene product (protein). Influenza virus genome encodes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2). A section of the influenza virus RNA encoding a particular protein can be referred to as “gene” or “gene segment.” The HA, NA, M1, and M2 are membrane associated, whereas NP, PB1, PB2, PA, and NS2 are nucleocapsid associated proteins. The HA and NA proteins are envelope glycoproteins, responsible for virus attachment and penetration of the viral particles into the cell, and the sources of the major immunodominant epitopes for virus neutralization and protective immunity. Each influenza virus subtype has mutated into a variety of strains with differing pathogenic profiles. Influenza A viruses are classified into subtypes based on antibody responses to HA and NA. There are 16 H and 9 N subtypes known, but only H 1, 2 and 3, and N 1 and 2 are commonly found in humans. Current subtypes of influenza A viruses found in people are seasonal influenza A (H1N1) and influenza A (H3N2) viruses. In the spring of 2009, a novel influenza A (H1N1) virus emerged to cause illness in people, which was very different from the human seasonal influenza A (H1N1) viruses circulating at that time. The novel virus often called “2009 pandemic H1N1” replaced the seasonal H1N1 virus that was previously circulating in humans. Influenza B viruses are not divided into subtypes, but can be further broken down into two lineages: B/Yamagata and B/Victoria. This document follows an internationally accepted naming convention for influenza viruses, as published in February 1980 in the Bulletin of the World Health Organization, 58(4):585-591 (1980). This convention uses the following components: the antigenic type (A, B, C); the host of origin (swine, equine, chicken, etc.; for human-origin viruses, no host of origin designation is given)”; geographical origin (Denver, Taiwan, etc.); strain number (15, 7, etc.); year of isolation (57, 2009, etc.); for influenza A viruses, the hemagglutinin and neuraminidase antigen description in parentheses (H1N1), (H5N1)). For example: A/Ann Arbor/06/1960-ca; A/Leningrad/134/1957-ca, B/Ann Arbor/01/1966-ca; B/USSR/60/1969-ca. In this document, pathogenic circulating influenza virus strains can be referred to as “circulating strains” or “community-acquired strains,” to distinguish them from influenza virus strains not currently in circulation, such as those used in live attenuated influenza vaccines.

The term “isolated” can be used in this document to refer to a biological component (such as a nucleic acid or a virus) that has been substantially separated or purified away from other biological components (such as cell debris, or other proteins or nucleic acids). Biological components that have been “isolated” include those components purified by standard purification methods. The term also embraces recombinant nucleic acids and viruses, as well as chemically synthesized nucleic acids.

Live Attenuated Influenza Vaccine (LAIV) is a trivalent or quadrivalent preparation, containing three or four live, cold-adapted (ca), temperature-sensitive (ts), attenuated influenza viruses: two influenza type A strains [subtype H3N2 and 2009 pandemic H1N1 (H1N1pdm09) and one or two influenza type B strain. Each of the LAIV viruses is a 6:2 genetic reassortment virus, containing the HA and NA gene segments from the recommended influenza vaccine virus and six internal gene segments derived from cold-adapted (ca) attenuated viruses, such as A/Ann Arbor/06/1960-ca or A/Leningrad/134/1957-ca-ca (LAIV-A virus strain) or B/Ann Arbor/01/1966-ca or B/USSR/60/1959-ca (LAIV-B strain). LAIV is typically administered intranasally. One example of LAIV is a vaccine manufactured by Medimmune (Gaithesburg, Md.) and sold under the trade names FluMist® and Fluenz®. Influenza viruses in LAIV are attenuated but living and may cause an infection with complications, for example, in people with weakened immune systems or other underlying medical conditions. Individuals receiving LAIV may also shed small amounts of the vaccine viruses for a period of time (a few days to weeks) after inoculation and may exhibit respiratory symptoms without experiencing a full-blown influenza infection, because LAIV virus may replicate in the nasopharynx for several days after inoculation

“Moiety” refers to a part or functional group of a molecule.

“Oligonucleotide” and the related terms are used in this document to refer to nucleic acid molecules, such as RNA or DNA molecules or their modifications, 200 bases long or less. The term “oligonucleotide” can refer to naturally occurring or non-natural (synthetic) nucleic acid sequences, as well as to the sequences containing residues, liners, labels etc. that do not naturally occur in nucleic acids, including modified natural nucleotides, nucleotides, etc.

“Primers” (singular—“primer”) are strands of short nucleic acid sequences, such as a DNA oligonucleotides, used as starting points for DNA synthesis during nucleic acid amplification reaction, such as PCR. Primers contain oligonucleotides with a sequences that can be annealed to a complementary target nucleic acid molecule by nucleic acid hybridization to form a hybrid between the primer and the target nucleic acid strand. A primer can be described as “specific” for a target nucleic acid. During the amplification reaction, a primer can be extended along the target nucleic acid molecule by a polymerase enzyme. Thus, primers can be used to amplify a target nucleic acid molecule (such as a portion of an influenza virus nucleic acid), wherein the sequence of the primer is specific for the target nucleic acid molecule, for example so that the primer will hybridize to the target nucleic acid molecule under high or very high stringency hybridization conditions employed in some parts of the PCR cycle. Primers are often characterized by “Primer Melting Temperature” (Tₘ), which is the temperature at which one half of the DNA duplex will dissociate to become single stranded and indicates the duplex stability. Primer melting temperature depends, in part, on its length and nucleotide sequence. A primer according to the embodiments of the present invention can be is at least 15 nucleotides in length, such as at least 15 contiguous nucleotides complementary to a target nucleic acid molecule, including the primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-60 nucleotides, 15-50 nucleotides, 20-40 nucleotides, or 15-30 nucleotides. Primers are generally used in pairs for amplification of a nucleic acid sequence, for example, by PCR, real-time PCR, or other nucleic-acid amplification methods. An “upstream” or “forward” primer is a primer 5′ to a reference point on a nucleic acid sequence. A “downstream” or “reverse” primer is a primer 3′ to a reference point on a nucleic acid sequence. At least one forward and one reverse primer are included in an amplification reaction. Primers can contain one or more detectable labels or reporters, meaning moieties that are detectable by various methods or assist in detection. In this case, primers act as probes during detection. For example, so-called scorpion primers can be used for detection in real-time PCR assays. Scorpion primers contain a stem-and-loop oligonucleotide structures with a 5′ fluorescent report and a 3′ quencher (“probe sequence”), which is attached to 5′ terminus of the oligonucleotide specific for the target nucleic acid sequence. During the annealing phase of the PCR, the probe sequence, the probe sequence hybridizes to the newly formed complementary target sequence, separating the fluorophore and the quencher dyes and leading to emission of fluorescence signal.

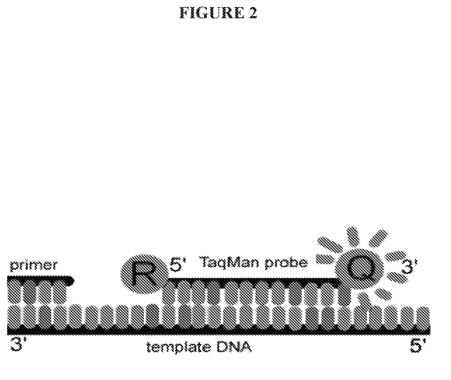

The term “probe” and the related terms are used in this document to refer to a molecule containing an oligonucleotide of variable length that is capable of hybridizing to a target nucleic acid sequence. The probe can be described as “specific for” the target nucleic acid sequence. Probes can be characterized by their Tₘ. The probes according to the embodiments of the present invention include rRT-PCR probes, which are probes capable of hybridizing to rRT-PCR amplification products. A probe can contain one or more detectable labels or reporters, meaning moieties that are detectable by various methods or assist in detection. For example, a variation of the probes described in this document are fluorescent reporter probes useful in rRT-PCR assays. One example of such probes are the so-called hydrolysis probes, such TaqMan® probes. TaqMan® probes are oligonucleotide probes that contain a covalently attached to 5′ end fluorescence reporter moiety and a quencher moiety, which can be attached at 3′ end or at an internal nucleotide, which reduces the fluorescence emitted by the fluorescent reporter. FIG. 2 schematically illustrates a TaqMan® probe (R denotes a reporter; Q denotes a quencher). Some examples of fluorophores that are suitable for use as fluorescent reporter dyes in TaqMan® probes are 6-carboxyfluorescein (FAM), tetrachlorofluorescein (TET), hexacholoro-6-carboxyfluorescein (HEX). When a probe is intact, the quencher suppresses the fluorescence of the fluorescence report dye. When the probe is used in real-time PCR, during the extension phase, the probe is cleaved by the exonuclease activity of the DNA polymerase, releasing the fluorophore. The fluorophore release results in an increase in fluorescence signal, which is proportionate to the amount of the PCR product.

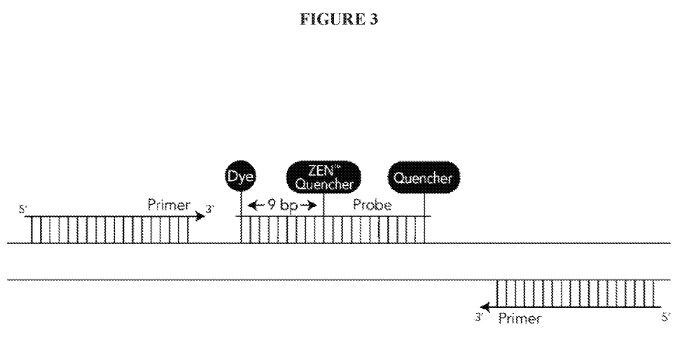

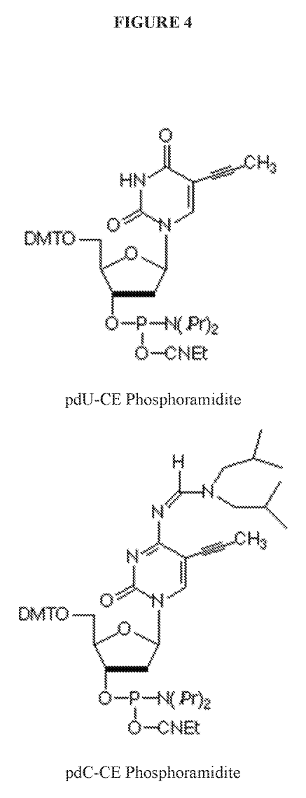

Variations and modifications of hydrolysis probes are possible. One example of such a modification is incorporation into a probe of conjugated Minor Groove Binder (MGB) groups, which act as duplex stabilizers. MGB probes typically incorporate a 5′ reporter dye and a 3′ nonfluorescent quencher, with the MGB moiety attached to the quencher molecule. One example of a MGB moiety is dihydrocyclopyrroloindole tripeptide (DPI₃), which folds into the minor groove formed by the terminal 5-6 bp of the probe. Such probes form extremely stable duplexes with single-stranded DNA targets, allowing shorter probes to be used. In comparison with unmodified DNA, MGB probes had higher melting temperature (Tₘ) and increased specificity. Another example is incorporation of modified bases, such as propyne derivatives, into a nucleotides. For example, substitution of C-5 propynyl-dC (pdC) for dC and C-5 propynyl-dU (pdU) for dT (both illustrated in FIG. 4) are effective strategies to enhance base pairing. These base substitutions enhance duplex stability and increase probe T^{m} by the following amounts: C-5 propynyl-C—2.8° C. per substitution; C-5 propynyl-U—1.7° per substitution. So-called BHQplus® provided by Biosearch technologies employ pdC and pdU substitutions in combination with BHQ dark quenchers. BHQplus and MGB probes can be used with oligonucleotides of shorter length and thus achieve an enhanced target specificity Another example of the probes used in real-time PCR assays are dual hybridization probes, which employ fluorescence resonance energy transfer (FRET) between the fluorophores on two different probes. Two fluorophore-labeled sequence-specific probes are designed to bind to the PCR product during the annealing phase of PCR, which results in an energy transfer from a donor fluorophore to an acceptor fluorophore. This results in an increase in fluorescence during the annealing phase. Some other examples of suitable probes are ZEN® Double-Quenched Probes (manufactured by Integrated DNA Technologies, Coraville, Iowa) (illustrated in FIG. 3) and QSY® probe from ThermoFisher Scientific, Waltham, Mass.

The terms “sample” or “samples,” as used interchangeably herein, include samples originating from human or animal subject (such as, but not limited to, samples of human or animal cells, tissues or bodily fluids and excretions) as well as samples prepared or generated by various laboratory and industrial processes, such as samples of virus isolates and vaccine samples. A sample can be directly obtained from a human or animal organism, obtained from the environment (such as food samples, water samples, surface swabs) propagated, cultured or synthesized. For example, a sample can be a virus isolate, including a primary isolate from a sample obtained from an infected individual, or an isolate propagated in the laboratory or industrially using various techniques, including recombinant techniques, tissue culture, propagation in eggs or nonhuman animals. Samples can be subject to various purification, storage or processing procedures before being analyzed according to the methods described herein. Generally, the terms “sample” or “samples” are not intended to be limited by their source, origin, manner of procurement, treatment, processing, storage or analysis, or any modification.

The term “section” or “region,” can be used in this document to refer to a part of a nucleic acid sequence, a part of a gene, or a part of a segment of influenza virus nucleic acid, and can be used interchangeably with the term “sequence.” For example, a section may be a part of PB1 gene of influenza virus derived from attenuated cold-adapted influenza virus A/Ann Arbor/06/1960-ca or A/Leningrad/134/1957-ca (LAIV-A PB1 gene), or a part of PA gene of LAIV-B virus derived from attenuated cold-adapted influenza virus B/Ann Arbor/01/1966-ca or B/USSR/60/1969-ca (LAIV-B PA gene). The examples of the regions of nucleic acid sequences utilized in the embodiments of the present invention is a region located at nucleotide positions 631-802 of PB1 gene of A/Ann Arbor/06/1960-ca (in reference to the sequence having Genbank accession number CY125908, incorporated herein by reference) and a region located at nucleotide position 889-1049 of PA gene segment of B/Ann Arbor/1/1966-ca (in reference to the sequence having Genbank accession number M20171, incorporated herein by reference).

The terms “sensitivity” and “specificity” can be used to refer to statistical measures of the performance of assays and methods described in this documents. Sensitivity refers to a proportion of positive results which are correctly identified by a test. Specificity measures a proportion of the negative results that are correctly identified by a test. Examples of the calculations used to determine specificity and specificity are below. Limit of detection (LOD) can also be used as a measure of sensitivity. Limit of detection can be expressed as a concentration of analyte, expressed in appropriate units, denoting a minimum concentration that can be detected by an assay or a detection method.

sensitivity=(number of samples determined as positive by rRT-PCR assay)/(samples determined as positive by the standard test, such as sequence analysis)

specificity=(number of samples negative by rRT-PCR assay)/(samples determined as negative by the standard test, such as sequence analysis)

The term “sequence” can be used to refer to the order of nucleotides in a nucleic acid, which can also be described as “primary structure,” or to a nucleic acid molecule, such as an oligonucleotide,” with a particular order of oligonucleotides.

“Sequence identity” or “sequence similarity” in the context of two or more nucleic acids sequences, refer to two or more sequences or subsequences that are the same or have a specified percentage nucleotides that are the same (for example, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region. Various tools for measuring sequence similarity are available, such as a BLAST or BLAST 2.0 sequence comparison algorithms with default parameters available from the National Center for Biological Information (NCBI) or other sources. See also Altschul et al., Nuc. Acids Res. 25:3389-3402 (1977) and Altschul et al., J. Mol. Biol. 215:403-410 (1990). For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Target nucleic acid” is a nucleic acid molecule or sequence intended for one or more of amplification, detection, quantitation, quantitative, semi-quantitative or qualitative detection. The nucleic acid molecule need not be in an isolated form purified form. Various other nucleic acid molecules can also be present with the target nucleic acid molecule. For example, the target nucleic acid molecule can be a specific nucleic acid molecule, which can include RNA (such as viral RNA) or DNA (such as DNA produced by reverse transcription of viral RNA). In the context of the embodiments of the present invention, a target nucleic molecule can be a LAIV nucleic acid molecule, such as a region of a LAIV gene.

The term “therapy” is used herein synonymously with the term “treatment.” The term “influenza therapy” as used herein encompasses various types of therapy or treatment, including drug therapy, palliative therapy, supporting therapy and symptomatic therapy.

The embodiments of the present invention use PCR methods to detect target nucleic acids of influenza virus. “Quantitative PCR” is a method that allows for quantification of the amounts of the target nucleic acid sequence used at the start at the PCR reaction. “Real-time PCR” is a method for detecting and measuring products generated during each cycle of a PCR, which are proportionate to the amount of template nucleic acid prior to the start of PCR. The information obtained, such as an amplification curve, can be used to determine the presence of a target nucleic acid (such as an influenza virus nucleic acid) and/or quantitate the initial amounts of a target nucleic acid sequence. In some examples, real-time PCR is real time reverse transcriptase PCR (rRT-PCR). Although some sources use the terms “real-time PCR” and “quantitative PCR” synonymously, this is not the case for the present document. Here, the term “quantitative PCR” encompasses all PCR-based techniques that allow for quantification of the initially present target nucleic acid sequences. The term “real-time PCR” is used to denote a subset of quantitative PCR techniques that allow for detection of PCR product throughout the PCR reaction, or in real-time. The principles of real-time PCR are generally described, for example, in Held et al. “Real Time Quantitative PCR” Genome Research 6:986-994 (1996). Generally, real-time PCR measures a signal at each amplification cycle. Some real-time PCR techniques rely on fluorophores that emit a signal at the completion of every multiplication cycle. Examples of such fluorophores are fluorescence dyes that emit fluorescence at a defined wavelength upon binding to double-stranded DNA, such as SYBR green. An increase in double-stranded DNA during each amplification cycle thus leads to an increase in fluorescence intensity due to accumulation of PCR product. Another example of fluorophores used for detection in real-time PCR are sequence-specific fluorescent reporter probes, described elsewhere in this document. The examples of such probes are TaqMan® probes. The use of sequence-specific reporter probe provides for detection of a target sequence with high specificity, and enables quantification even in the presence of non-specific DNA amplification. Fluorescent probes can also be used in multiplex assays—for detection of several genes in the same reaction-based on specific probes with different-colored labels. For example, a multiplex assay can use several sequence-specific probes, labeled with a variety of fluorophores, including, but not limited to, FAM, JA270, CY5.5, and HEX, in the same PCR reaction mixture.

Real-time PCR relies on detection of a measurable parameter, such as fluorescence, during the course of the PCR reaction. The amount of the measurable parameter is proportional to the amount of the PCR product, which allows observe the increase of the PCR product “in real time.” Some real-time PCR methods allow for quantification of the input DNA template based on the observable progress of the PCR reaction. The analysis and processing of the data involved is discussed below. A “growth curve” or “amplification curve” in the context of a nucleic acid amplification assay is a graph of a function, where an independent variable is the number of amplification cycles and a dependent variable is an amplification-dependent measurable parameter measured at each cycle of amplification, such as fluorescence emitted by a fluorophore. As discussed above, the amount of amplified target nucleic acid (such as an influenza nucleic acid) can be detected using a fluorophore-labeled probe. Typically, the amplification-dependent measurable parameter is the amount of fluorescence emitted by the probe upon hybridization, or upon the hydrolysis of the probe by the nuclease activity of the nucleic acid polymerase. The increase in fluorescence emission is measured in real time and is directly related to the increase in target nucleic acid amplification (such as influenza nucleic acid amplification). In some examples, the change in fluorescence (dRₙ) is calculated using the equation dRₙ=Rₙ₊−Rₙ₋, with Rₙ₊ being the fluorescence emission of the product at each time point and Rₙ₋ being the fluorescence emission of the baseline. The dRₙ values are plotted against cycle number, resulting in amplification plots. In a typical polymerase chain reaction, a growth curve comprises a segment of exponential growth followed by a plateau, resulting in a sigmoidal-shaped amplification plot when using a linear scale. A growth curve is characterized by a “cross point” value or “Cₚ” value, which can be also termed “threshold value” or “cycle threshold” (Cₜ), which is a number of cycles where a predetermined magnitude of the measurable parameter is achieved. For example, when a fluorophore-labeled probe is employed, the threshold value (Cₜ) is the PCR cycle number at which the fluorescence emission (dRₙ) exceeds a chosen threshold, which is typically 10 times the standard deviation of the baseline (this threshold level can, however, be changed if desired). A lower Cₜ value represents more rapid completion of amplification, while the higher Cₜ value represents slower completion of amplification. Where efficiency of amplification is similar, the lower Cₜ value is reflective of a higher starting amount of the target nucleic acid, while the higher Cₜ value is reflective of a lower starting amount of the target nucleic acid. Where a control nucleic acid of known concentration is used to generate a “standard curve,” or a set of “control” Cₜ values at various known concentrations of a control nucleic acid, it becomes possible to determine the absolute amount of the target nucleic acid in the sample by comparing Cₜ values of the target and control nucleic acids.

Assays

Embodiments of the present invention include real-time RT-PCR (rRT-PCR) assays useful for detection of certain influenza viruses used in the production of LAIV. The assays according to the embodiments of the present invention can detect a section of PB1 gene of an influenza virus derived from attenuated cold-adapted influenza virus A/Ann Arbor/06/1960-ca or A/Leningrad/134/1957-ca (“LAIV-A virus strain”). The PB1 gene of these two strains originated from an avian A/H2N2 influenza virus species and is genetically different from seasonal A/H1N1, seasonal A/H3N2 and 2009 pandemic A/H1N1 influenza viruses. Accordingly, a region located in PB1 gene can be used to differentiate between seasonal influenza A virus strains and a LAIV-A virus strain. The assays according to the embodiments of the present invention can also detect a region of PA gene of LAIV-B virus derived from attenuated cold-adapted influenza virus B/Ann Arbor/01/1966-ca or B/USSR/60/1969-ca (LAIV-B strain) that may be used for differentiating between seasonal influenza B virus strains and a LAIV-B virus strain. The examples of the regions of nucleic acid sequences utilized in the embodiments of the present invention is a region located at nucleotide positions 631-802 of PB1 gene of A/Ann Arbor/06/1960-ca (in reference to the sequence having Genbank accession number CY125908) or a region with a corresponding sequence of A/Leningrad/134/1957-ca PB1 gene, and a region located at nucleotide position 889-1049 of PA gene segment of B/Ann Arbor/1/1966-ca (in reference to the sequence having Genbank accession number M20171) or a corresponding sequence of B/USSR/60/1969-ca PA gene. The assays according to the embodiments of the present invention can detect the above gene regions with a high degree of specificity and sensitivity.

The specificity of the assays according to the embodiments of the present invention allows them to discriminate between influenza virus strains that contain one of the above gene regions, which are influenza virus strains typically used in LAIV, and circulating influenza virus strains, which currently do not contain one of the above gene regions. When the assays of the present invention are used to detect LAIV, the specificity of the assays according to the embodiments of the present thus minimizes or avoids false positive assay results generated by other types LAIV assays, which often cross-react with circulating influenza strains. The sensitivity of the assays according to the embodiments of the present invention allows them to detect low amounts of LAIV virus strains, thus minimizing or avoiding false negative assay results.

rRT-PC assays of the present invention are designed to detect a region of PB1 gene of LAIV-A virus strain (“LAIV-A assay”) and a region of PA gene of LAIV-B (“LAIV-B assay”) virus strain using one or more DNA primers and probes based on the sequences listed in Table 1. It is to be understood, of course, that the uses of assays of the present invention, as well as of the primers and the probes described in this documents, are not limited to the detection of LAIV virus strains. The assays, the primers and the probes can be used to detect any influenza virus or, more generally, the nucleic acids containing the above gene regions or one or more the relevant sequences used in the primer and/or probe design (shown in Table 1).

TABLE 1
Primer and probe sequences

| | | DNA Oligonucleotide |
|---|---|---|
| Name | SEQ ID NO | Sequence (5′-3′) |
| LAIV-A | SEQ ID NO: 1 | GAA GCA AAG ATT GAA |
| forward primer | | CAA GAG AAG C |
| LAIV-A | SEQ ID NO: 2 | CAC AAA TAC TTC TCG |
| reverse primer | | CTA GTG TTT CG |
| LAIV-A probe | SEQ ID NO: 3 | CAC TGA CAT T GAA CAC |
| | | AAT GAC TAA AGA TGC |
| LAIV-B | SEQ ID NO: 4 | ATG AGT TGG GGC TGG |
| forward primer | | CTA |
| LAIV-B | SEQ ID NO: 5 | CCA CAG CTT CCA TAA |
| reverse primer | | GAA GT |
| LAIV-B Probe | SEQ ID NO: 6 | TTC TTG GAC TTC CCT |
| | | TCA G |

The specificity of the assays according to the embodiments of the present invention can be at least about 95%, 96%, 97%, 98%, or about 95%, 96%, 97%, 98%, or 100%. The sensitivity of the assays according to the embodiments of the present invention can be at least about 95%, 96%, 97%, 98%, or about 95%, 96%, 97%, 98%, or 100%. The detection limit of the assays according to the embodiments of the present invention can be about 10^{1.3}, 10^{1.4}, 10^{1.3}, 10^{1.5}, 10^{1.6}, 10^{1.6}, 10^{1.7}, 10^{1.8}, 10^{1.9}, 10^{2.0}, 10^{2.1}, 10^{2.2}, 10^{2.3}, 10^{2.4}, 10^{2.5}, 10^{2.6}, or less than any of the above values denoting Tissue Culture Infectious Doses per milliliter (TCID₅₀/ml) or Egg Infectious Doses per milliliter (EID₅₀/ml).

The assays according to the embodiments of the present invention can serve as effective tools for rapid identification of LAIV viruses in clinical and laboratory samples with high sensitivity and specificity. The assays of the present invention can have various application and uses. For example, LAIV assays can be used to test specimens collected from individuals with respiratory symptoms who were recently immunized with LAIV to determine if a LAIV virus may have been a causative agent. LAIV assays can also be employed to test LAIV vaccine samples to verify their identity and the titer of LAIV virus strains.

Probes

Some embodiments of the present invention are oligonucleotide probes that can be employed to detect LAIV virus strains in rRT-PCR assays. The probes of the present invention are designed to detect a region of PB1 gene of LAIV-A virus strain (“LAIV-A probe”) and a region of PA gene of LAIV-B virus strain (“LAIV-B probe”) and are based on the sequences listed in Table 1. The probes are not limited to LAIV detection and can be used to detect any influenza virus nucleic acids or other nucleic acids containing the sequences used in the probe design (shown in Table 1).

The embodiments of the present invention include DNA probes suitable for detection of a region of PB1 gene of LAIV-A virus, which contain a sequence at least 90% identical to SEQ ID NO:3. Some embodiments of the probe contain an oligonucleotide of SEQ ID NO:3. Some other embodiments consist of an SEQ ID NO:3 and reporting moieties discussed below. The length on the probe depends on the primers selected for a particular rRT-PCR assay. The probe is designed with a Tₘ 8-10° C. higher than Tₘ of the primer. For example, a probe, such as LAIV-A probe can be about 30 bp long (meaning 30±3, 30±2, 30±1 bp long, for example, the probe can be 27-33 bp, 20-33 bp, or 20-30 bp long). The probe can be a TaqMan, problem labeled with a fluorophore moiety and a fluorescence quencher moiety, but other types of probe chemistries can be employed. In an exemplary TaqMan®) probe, the fluorophore moiety is coupled to 5′ terminus of the probe. One example of a suitable fluorophore is a fluorescein moiety. One example of a suitable quencher is a dark quencher, for example BHQ quencher, such as BHQ1. The quencher can be coupled to 3′ terminus of the probe or to an internal base. The probe can also contain a duplex stabilizer. One exemplary embodiment of a LAIV-A probe is a probe consisting of SEQ ID NO:3 oligonucleotide, FAM fluorophore coupled to 5′ terminus and BHQ1 label coupled to an internal T residue of the oligonucleotide, as shown in Table 2. Some other examples of suitable probes are The ZEN®, Double-Quenched Probes (manufactured by Integrated DNA Technologies, Coraville, Iowa) and QSY® probe from ThermoFisher Scientific, Waltham, Mass. comprising SEQ ID NO:3 oligonucleotide. The above probes were evaluated in the assays according to the embodiments of the present invention and were found to perform comparably to TaqMan® probes.

Embodiments of the present invention also include a DNA probe suitable for detection of a region of the PA gene of LAIV-B virus, which contains a sequence at least 90% identical to SEQ ID NO:6. In one embodiment, the problem contains (comprises) SEQ ID NO:6. In another embodiment, the probe consists of SEQ ID NO:6 and the reporting moieties, such as a fluorescein moiety coupled to 5′ terminus of the probe and a dark quencher, such as BHQ1, coupled to 3′ terminus of the probe. In one example, LAIV-B probe is a probe containing modified bases for duplex stabilization, commercially available as BHQplus probe. As discussed earlier in this document, BHQplus and MGB probes form highly stable duplexes with DNA targets, allowing shorter probes to be used for hybridization-based assays. Use of shorter probes can improve the ΔTₘ of a mismatch assay and achieved enhanced target specificity. LAIV-B BHQplus probe is designed with a Tm 8-10° C. higher than Tₘs of the primers. In a particular embodiment, the probe is about 19 bp long (for example, 19±3, 19±2 or 19±1 bp long, for example, the probe can be 16-22 bp long). LAIV-B BHQplus probe comprises SEQ ID NO:6 oligonucleotide with each T substituted for pdU and each C substituted for pdC, FAM label at the 5′ end and BHQ1 quencher at the 3′ end, as shown in Table 2. Another example of a LAIV-B probe is a probe containing a duplex stabilizer, such as a commercially available MGB probe.

Primers

Embodiments of the present invention include DNA oligonucleotides that can be employed for amplification of LAIV virus sequences. However, the primers described in this document are not limited to LAIV amplification and can be used to amply any influenza virus nucleic acids or other nucleic acids containing the sequences used in the probe design (shown in Table 1). Among the embodiments of the present invention are primers suitable for amplification of a region of the PB1 gene of LAIV-A virus strain (“LAIV-A primers”). LAIV-A primers include a “forward” primer comprising an oligonucleotide at least 90% identical SEQ ID NO:1 (“LAIV-A forward primer”) and a “reverse” primer comprising an oligonucleotide at least 90% identical SEQ ID NO:2 (“LAIV-A reverse primer”). For example, a LAIV-A forward primer can be an oligonucleotide comprising SEQ ID NO: 1, an oligonucleotide consisting of SEQ ID NO:1, or oligonucleotide consisting of SEQ ID NO:1 and reporting moieties or labels. A LAIV-A reverse primer can be an oligonucleotide comprising SEQ ID NO:2, an oligonucleotide consisting of SEQ ID NO:2, or oligonucleotide consisting of SEQ ID NO:2 and reporting moieties or labels. It is understood that, for amplification of region of the PB1 gene of LAIV-A virus strain, the primers can be used together as a primer pair, but can also be used separately in combination with the other primers. For example, LAIV-A forward primer can be combined with LAIV-A reverse primer for amplification of PB1 gene region, but can also be combined with a suitable reverse primer other than LAIV-B. Likewise, LAIV-A reverse primer can be combined with LAIV-A forward primer for amplification of PB1 gene region, but can also be combined with a reverse primer other than LAIV-A. The length of the primers can vary. For example, LAIV-A primers can be 23-30 bp long, for example, 23, 24, 35, 26, 27, 28, 29, 30, 31 and 32 bp long.

Also among the embodiments of the present invention are primers for amplification of a region of the PA gene of LAIV-B virus strain (“LAIV-B primers”). LAIV-B primers include a “forward” primer comprising a DNA or modified DNA oligonucleotide at least 90% identical to SEQ ID NO:4 (“LAIV-B forward primer”) and a “reverse” primer comprising a DNA or modified DNA oligonucleotide at least 90% identical SEQ ID NO:5 (“LAIV-B reverse primer”). For example, LAIV-B forward primer can be an oligonucleotide comprising SEQ ID NO:4, an oligonucleotide consisting of SEQ ID NO:4, or oligonucleotide consisting of SEQ ID NO:4. LAIV-B reverse primer can be an oligonucleotide comprising SEQ ID NO:5, an oligonucleotide consisting of SEQ ID NO:5, or oligonucleotide consisting of SEQ ID NO:5. It is understood that, for amplification of a region of the PA gene of LAIV-B virus strain, LAIV-B forward and reverse primers can be used together as a primer pair, but can also be combined with other primers. For example, LAIV-B forward primer can be combined with LAIV-B reverse primer for amplification of A gene region, but can also be combined with a suitable reverse primer other than LAIV-B reverse primer. Likewise, LAIV-B reverse primer can be combined with LAIV-B forward primer for amplification of PA gene region, but can also be combined with a reverse primer other than LAIV-A. The length of the primers can vary. For example, LAIV-B primers can be 16-30 bp long, 16-25 bp long, or 16-20 bp long, for example, 16, 17, 18, 19, 20, 21, 22, 23, 24, 25, 26, 27, 28, 29, 30, 31 and 32 bp long.

It is to be understood that the primers according to the embodiments of the present invention can be unmodified and unlabeled DNA oligonucleotides. The primers according to the embodiments of the present invention can also contain reporting or labelling moieties, such as fluorescent moieties, quencher moieties or their combinations. The primers according to the embodiments of the present invention can also contain unnatural and modified nucleotides, linkers and other moieties.

Kits

The embodiments of the present invention also include kits comprising one or more of the primers and the probes described above. In other words, the primers according to the embodiments of the present invention can be included or combined, in various ways, in kits. Such kits can be used for detection, including semi-quantitative and quantitative detection, of LAIV-A, LAIV-B, or both LAIV-A and LAIV-B virus strains in samples, such as the samples derived from human subjects, laboratory samples, virus isolate samples or vaccine samples. It is to be understood that at least some of the kits described in this document are not limited to LAIV amplification or detection and can be used to detect and/or amplify any influenza virus nucleic acids or other nucleic acids containing the sequences used in the design or the probes included in the kits. These sequences are shown in Table 1.

Some examples of the kit embodiments are described below. LAIV-A, LAIV-B probes or both LAIV-B and LAIV-A probes discussed in the section can be included in the kits useful for detecting LAIV virus strains by rRT-PCR assays. For example, a LAIV-A probe described in the section “Probes” can be included in a kit along with other reagents for performing a rRT-PCR assay. Such a kit can be used for detecting LAIV-A virus strain in the sample. In another example, a LAIV-B probe described in the section “Probes” can be included in a kit along with other reagents for performing an rRT-PCR assay. Such a kit can be used for detecting LAIV-B virus strain in the sample. In one more example, a LAIV-A probe and a LAIV-B probe can be included in a kit along with other reagents for performing a rRT-PCR assay. Such a kit can be used for detecting LAIV-A, LAIV-B or both in the sample.

The other reagents included in the kits can include LAIV-A and LAIV-B primers. For example, a kit can include a LAIV-A probe and one or both of LAIV-A forward primer and LAIV-A reverse primer. In another example, a kit can include a LAIV-B probe and one or both of LAIV-B forward primer and LAIV-B reverse primer. In one more example, a kit can include a LAIV-B probe, a LAIV-B probe, one or both of LAIV-A forward primer and LAIV-A reverse primer, and one or both of LAIV-B forward primer and LAIV-B reverse primer.

The kits can include additional reagents for performing an rRT-PCR assay. The examples of additional reagents are enzymes for performing rRT-PCR assays are reverse transcriptase, DNA polymerase, such as Taq polymerase, PCR buffers, dNTPs and various additives, such as the additives that allow for efficient amplification of GC-rich templates. Some other examples of possible additional reagents are DNA-binding dyes, such as SYBR Green, which can be employed in rRT-PCR assays that employ unlabeled primers and no probes.

Methods

Embodiments of the present invention also include methods of using the primers, probes and kits described above (“method embodiments”). Some of the method embodiments are methods of amplifying a region of a PB1 gene of LAIV-A virus strain and a PHI gene of LAIV-B virus strain by a PCR using one or more of the primers described in section “Primers” of this document. Such methods can be referred to as “methods of amplifying a LAIV virus strain,” “amplification methods,” and by other related expressions and include a step of contacting a sample, which may contain a LAIV-A or a LAIV-B virus strain, with one or more primers described in the section primers. When the goal of the method is amplifying a region of a PB1 gene of LAIV-A virus strain, a forward LAIV-A primer, a reverse LAIV-A primer, or a combination of a forward and a reverse LAIV-A primer is employed. When the goal of the method of the method is amplifying a region of a PB1 gene of LAIV-A virus strain, a forward LAIV-A primer, a reverse LAIV-A primer, or a combination of a forward and a reverse LAIV-A primer is employed. It is to be understood that both LAIV-A and LAIV-B primers can be employed in some embodiments of the amplification methods. After the contacting step, a PCR (such as rRT-PCR, discussed in more detail elsewhere in this document) is performed under suitable conditions and using suitable reagents, and the amplification products can be detected by various detection procedures. The amplification methods can be used to determine if LAIV-A and/or LAIV-B virus strain is present in the sample based on the detection of one or more products of the amplification.

Some of the method embodiments rely on detection of a gene region of PB1 gene of LAIV-A virus strain and/or detection of a gene region of PA gene of LAIV-B virus strains using the probes according to the embodiment of the present invention in a rRT-PCR assay. One example of a method embodiment is a method of detecting a presence or absence of a LAIV-A virus strain in a sample. This method embodiment includes a step of contacting the sample with one of the LAIV-A probes described in the section “Probes,” and forward and reverse primers specific for at least one nucleic acid sequence of the PB1 gene region of LAIV-A for which the probe is specific. A forward primer may be one of the LAIV-A primers described in this document. A reverse primer may be one of the LAIV-A primers described in this document.

In the detection methods that employ rRT-PCR (rRT-PCR methods or assays), rRT-PCR is performed under suitable conditions and using suitable reagents following the contacting step in order to generate a PCR cycle threshold, and this cycle threshold is compared to a control value. In a semi-quantitative variation of rRT-PCR methods, if the cycle threshold is below the control value, the LAIV virus strain is absent from the sample, and if the cycle threshold is above the control value, the LAIV virus strain is present in the sample. An example of a cycle threshold control (cutoff) value is Cₜ value of CDC Human Influenza Real-Time RT-PCR Diagnostic Panel, Cₜ=38, same here. In a quantitative variation of rRT-PCR methods, the method can include a step of determining a quantity of the LAIV virus strain when the LAIV virus strain is present in the sample.

The calculations and comparisons (e.g., of a sample signal to a control value or range) for the methods described herein can involve computer-based calculations and tools. Tools can be advantageously provided in the form of computer programs that are executable by a general purpose computer system (which can be called “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives. CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java. JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

The amplification and the detection methods of the present invention can have various application. For example, they can be used in a method of determining if a patient is infected with or has been exposed to a LAIV virus strain having a gene region from PB1 gene of LAIV-A virus strain or the PA gene of LAIV-B virus strain. Such a method can be employed as a diagnostic method in a clinical setting, for example, to make the decisions about the treatment of the patients. For example, administration of LAIV to healthy children from 2 until 8 years of age was recommended by the U.S. Center for Disease Control, when available. The administration of LAIV may lead to higher false positive diagnostic influenza tests, resulting in possible misdiagnosis. Thus, it is important to consider vaccination history when interpreting positive influenza diagnostic test results, particularly early in the influenza season. Further, if positive test results are suspected to be due to LAIV, influenza may not be a causative agent for illness and testing for other pathogens should be considered. The methods of the present invention can also be employed in influenza surveillance efforts. For example, testing of a collection of the samples obtained from a population using the methods of the present invention in addition to the testing of the same sample collection for circulating influenza virus strains can generate more accurate epidemiological data on the prevalence of influenza virus infection in a population.

The amplification and the detection methods of the present invention can also be used for quality control of LAIV. For example, LAIV samples, particularly, but not limited, those originating from suspect sources or suspected of being exposed suboptimal storage or production conditions, can be tested to verify the identify the presence and the amounts of LAIV virus strains found in the vaccines.

### EXAMPLES

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### rRT-PCR Assay for Detection of LAIV Vaccine

Detection of LAIV vaccine by rRT-PCR assay was performed using rRT-PCR reaction conditions consistent with the current CDC Human Influenza Viruses rRT-PCR Diagnostic Panel (“CDC Flu rRT-PCR Dx Panel”) for the detection and sub-typing of influenza viruses. Probes and primers employed in the assay are shown in Table 2.

TABLE 2
Pruners and probes for detection of LAIV-A and
LAIV-B sequences by rRT-PCR assay

| | | Oligonucleotide | |
|---|---|---|---|
| Name | SEQ ID NO | Sequence (5′-3′) | Label |
| LAIV-A | SEQ ID NO: 1 | GAA GCA AAG ATT | None |
| Forward | | GAA CAA GAG AAG | |
| Primer | | C | |
| LAIV-A | SEQ ID NO: 2 | CAC AAA TAC TTC | None |
| Reverse | | TCG CTA GTG TTT | |
| Primer | | CG | |
| LAIV-A | SEQ ID NO: 3 | CAC TGA CAT T | FAM at 5′ end, |
| Probe | | GAA CAC AAT GAC | BHQ1 at the |
| | | TAA AGA TGC* | internal 10^{th} |
| | | | T base, shown |
| | | | in bold |
| LAIV-B | SEQ ID NO: 4 | ATG AGT TGG | None |
| Forward | | GGC TGG CTA | |
| primer | | | |
| LAIV-B | SEQ ID NO: 5 | CCA CAG CTT | None |
| Reverse | | CCA TAA GAA GT | |
| primer | | | |
| LAIV-B | SEQ ID NO: 6 | TTC TTG GAC TTC | FAM at 5′ end, |
| Probe | | CCT TCA G** | BHQ1 at 3′ |
| | | | end*** |

*The spacer was added at 3′ end to prevent probe extension by Taq polymerase; the quencher was added internally to improve real time PCR perfomiance. ZEN ® Double-Quenched Probes and QSY ® probes were also tested.
**BFIQplus probe was employed with each T substituted by pdU and each C substituted by pdC.
**TaqMan ®MGB Probe was also used, which incorporated a 5′ reporter dye, a 3′ nonfluorescent quencher (NFQ), with the MGB moiety attached to the quencher molecule.

Viral RNA was isolated from 100 μl of clinical specimens, LAIV vaccine or virus isolates and eluted into a final volume of 100 μl elution buffer using MagNA Pure® Compact RNA isolation kit on a MagNA Pure® Compact instrument (Roche Applied Sciences, Indianapolis, Ind.) rRT-PCR reactions were performed using Invitrogen SuperScript® III Platinum® One-Step qRT-PCR kit (Life Technologies, Carlsbad, Calif.) (“Invitrogen kit”), Ambion AgPath-ID® One-Step RT PCR kit (Ambion, Austin, Tex.) (“AgPath kit”), and qScript™ One-Step qRT-PCR kit (Quanta Biosciences, Gaithersburg, Md.) (“Quanta kit”). The starting composition of the PCR reaction mixtures are shown in Table 3. Final primer and probe reaction concentrations at the start of the reaction were 0.8 μM and 0.2 μM, respectively. rRT-PCR reactions were carried out in a 7500 Fast Dx Real-Time PCR instrument (Applied Biosystems, Foster City, Calif.) or an MX3005 QPCR system (Stratagene, La Jolla, Calif.). rRT-PCR thermocycling conditions are shown in Table 4.

TABLE 3
rRT-PCR reaction conditions (total 25.0 μl)

| | Invitrogen | | Quanta |
|---|---|---|---|
| | kit | AgPath kit | kit |
| Nuclease free water | 5.5 μl | 5.0 μl | 5.5 μl |
| Forward primer, 40 μM | 0.5 μl | 0.5 μl | 0.5 μl |
| Reverse primer, 40 μM | 0.5 μl | 0.5 μl | 0.5 μl |
| Probe, 10 μM | 0.5 μl | 0.5 μl | 0.5 μl |
| SuperScript ™ III RT/Platinum ® | 0.5 μl | 1.0 μl | 0.5 μl |
| Taq Mix | | | |
| SuperScriptTM III RT/Platinum ® | 12.5 μl | 12.5 μl | 12.5 μl |
| 2X PCR Master Mix* | | | |
| RNA (Sample or control)** | 5.0 μl | 5.0 μl | 5.0 μl |

*consists of a proprietary buffer system, MgSO₄, dNTPs, and stabilizers. 50 mM MgSO4 may be added for further optimization of the Mg²⁺ concentration.
**FluMist LAIV vaccine was used as a control.

TABLE 4
rRT-PCR thermocycling conditions

| rRT-PCR reaction phase | Invitrogen kit | AgPath kit | Quanta kit |
|---|---|---|---|
| Reverse Transcription | 50° C. for 30 min | 50° C. for 30 min | 50° C. for 30 min |
| Taq inhibitor inactivation | 95° C. for 2 min | 95° C. for 10 min | 95° C. for 5 min |
| PCR amplification (45 | 95° C. for 15 sec | 95° C. for 15 sec | 95° C. for 15 sec |
| cycles) | 55° C. for 30 sec* | 55° C. for 30 sec* | 55° C. for 30 sec* |

*Fluorescence data was collected during the 55° C. incubation step.

### Example 2

### Detection of LAIV Vaccine Using rRT-PCR Assay

Analytical performance of the rRT-PCR assay described in Example 1 was evaluated by testing ten-fold serial dilutions of RNA extracted from 2010-2011 LAIV vaccine (FluMist®) The results are summarized in table 5. Analytical performance of both LAIV-A and LAIV-B rRT-PCR assays were comparable to the CDC Flu rRT-PCR Dx Panel universal influenza A (InfA) assay and universal influenza B (InfB) assay, respectively.

TABLE 5
Assay sensitivity on 2012 LAIV vaccine (FluMist ®) determined with the
Invitrogen SuperScript ™III Platinum ® One-Step quantitative RT-PCR Kits

| Serial | | | | |
|---|---|---|---|---|
| dilution of | Cₜ values for triplicate assays | | | |
| FluMist ® | InfA | LAIV-A | InfB | LAIV-B |
| 10⁻⁴ | 29.6/29.3/29.7 | 24.8/25.1/25.5 | 30.8/30.5/30.2 | 30.0/30.1/30.1 |
| 10⁻⁵ | 33.2/32.4/32.2 | 28.7/28.3/29.0 | 34.3/33.9/33.8 | 33.5/34.0/33.3 |
| 10⁻⁶ | 32.4/36.3/37.9 | 31.8/32.8/32.3 | 37.7/37.0/38.1 | 36.1/37.1/39.1 |
| 10⁻⁷ | —/—/— | 36.8/34.1/35.3 | —/—/— | 40.0/—/— |
| 10⁻⁸ | —/—/— | —/—/— | —/—/— | —/—/— |

### Example 3

### Testing of Virus Isolates of Circulating Virus Strains Using LAIV rRT-PCR Assay

TABLE 6
Analytical specificity testing of LAIV-A rRT-PCR assay
performed on the samples of seasonal A(H1N1), A(H3N2)
and A(H1N1)pdm09 influenza virus isolates

| | | | Cₜ Value | |
|---|---|---|---|---|
| | | Infectious | InfA | LAIV-A |
| Influenza Virus | Subtype | Titer | assay | assay |
| A/Brisbane/59/2007 | H1N1 | 8.4^{a} | 12.83 | — |
| A/Bangladesh/7286/2007 | H1N1 | 6.1^{b} | 13.53 | — |
| A/Beijing/262/1995 | H1N1 | 6.0^{b} | 11.71 | — |
| A/Fukushima/141/2006 | H1N1 | 5.7^{b} | 17.49 | — |
| A/Hawaii/15/2001 | H1N1 | 6.6^{a} | 15.67 | — |
| A/Jiangxi/160/2005 | H1N1 | 5.6 | 13.32 | — |
| A/South Dakota/06/2007 | H1N1 | 8.2^{a} | 13.73 | — |
| A/Mexico/1729/2007 | H1N1 | 4.8^{b} | 13.67 | — |
| A/New Caledonia/20/1999 | H1N1 | 6.6^{a} | 12.73 | — |
| A/Solomon Islands/03/2006 | H1N1 | 6.2^{b} | 15.12 | — |
| A/Perth/16/2009 | H3N2 | 8.2^{a} | 11.01 | — |
| A/Anhui/1239/2005 | H3N2 | 8.1^{b} | 11.94 | — |
| A/Afghanistan/2903/2008 | H3N2 | 5.0^{b} | 14.79 | — |
| A/Brisbane/10/2007 | H3N2 | 6.8^{b} | 11.72 | — |
| A/Hawaii/08/2006 | H3N2 | 7.8^{b} | 11.60 | — |
| A/New York/55/2004 | H3N2 | 6.4^{a} | 12.80 | — |
| A/Wisconsin/67/2005 | H3N2 | 6.5^{a} | 14.51 | — |
| A/Uruguay/716/2007 | H3N2 | 8.2^{a} | 11.78 | — |
| A/Taiwan/760/2007 | H3N2 | 5.5^{b} | 10.99 | — |
| A/British Columbia/ | H3N2 | 6.0^{b} | 12.77 | — |
| RV1287/2007 | | | | |
| A/California/04/2009 | H1N1pdm09 | 7.6^{b} | 14.07 | — |
| A/California/07/2009 | H1N1pdm09 | 8.4^{a} | 13.59 | — |
| A/New York/18/2009 | H1N1pdm09 | 7.8^{a} | 14.21 | — |
| Asia2007-1_clade 1.1 | H5N1 | 4.5^{a} | 27.11 | — |
| Asia2005-1_clade 2.1 | H5N1 | 8.5^{a} | 9.95 | — |

^{a}EID₅₀/ml;
^{b}TCID₅₀/ml

TABLE 7
Analytical specificity testing of LAIV-B rRT-PCR assay with
influenza B viruses

| | | Ct Value | |
|---|---|---|---|
| Influenza virus | Infectious Titer | InfB assay | LAIV-B assay |
| B/Brisbane/60/2008 | 8.5^{a} | 10.49 | — |
| B/Beijing/184/1993 | 3.0^{b} | 12.21 | — |
| B/Brisbane/03/2007 | 8.4^{a} | 11.37 | — |
| B/Florida/04/2006 | 6.4^{a} | 10.37 | — |
| B/Florida/07/2004 | 6.0^{a} | 13.15 | — |
| B/Ohio/01/2005 | 7.5^{a} | 12.38 | — |
| B/Pennsylvania/07/2007 | 8.2^{a} | 11.17 | — |
| B/Santiago/4364/2007 | 7.2^{a} | 13.71 | — |
| B/Texas/03/2007 | 5.6^{a} | 13.94 | — |
| B/Victoria/304/2006 | 8.2^{a} | 11.97 | — |

^{a}EID₅₀/ml;
^{b}TCID₅₀/ml

The specificity of LAIV-A and LAIV-B rRT-PCR assay described in Example 1 was evaluated using the samples of cultured virus isolates of the circulating influenza virus strains from CDC collection. The results of the testing are summarized in Tables 6 and 7. False positive results were not observed when testing of cultured human A(H1N1)pdm09, A(H1N1), A(H3N2) and B influenza viruses was performed.

### Example 4

### Testing of Clinical Specimens Using LAIV rRT-PCR Assay

Testing of clinical specimens using LAIV-A and LAIV-B rRT-PCR assays was performed. The clinical specimens were received from U.S state and local public health laboratories. The results of the testing are summarized in Tables 8A and 8B. It was determined that a total of forty-two human specimens collected in the U.S from January 2010 to January 2012 were positive for LAIV viruses, and results were confirmed by genetic sequence analysis. Of those forty-two cases, eleven cases were positive for LAIV-A, twenty-one cases were positive for LAIV-B, and ten cases were positive for both LAIV-A and LAIV-B. The LAIV-A assay showed no cross reactivity with human seasonal H1N1 and H3N2, 2009 pandemic H1N1 and H5N1 influenza A viruses; the LAIV-B assay showed no cross reactivity with wild type influenza B viruses.

TABLE 8
Summary of LAIV-A and LAIV-B rRT-PCR test results of
human respiratory specimens, in comparison to sequence analysis
A.

| | | Bi-directional Sequencing Result | | | |
|---|---|---|---|---|---|
| | | LAIV-A | LAIV-A | | |
| | | Positive | Negative^{a} | Total | Performance |
| LAIV-A | LAIV-A | 21/21 | 0/0 | 21 | Sensitivity |
| rRT-PCR | Positive | | | | 100% |
| Result | LAIV-A | 0/0 | 52/52 | 52 | Specificity |
| | Negative | | | | 100.0% |
| | Total | 21 | 52 | 73 | Performance |
| | | | | | Agreement |
| | | | | | 100.0% |

B.

| | | Bi-directional Sequencing Result | | | |
|---|---|---|---|---|---|
| | | LAIV-B | LAIV-B | | |
| | | Positive | Negative^{a} | Total | Performance |
| LAIV-B | LAIV-B | 31/31 | 0/0 | 31 | Sensitivity |
| rRT-PCR | Positive | | | | 100% |
| Result | LAIV-B | 0/0 | 46/46 | 46 | Specificity |
| | Negative | | | | 100.0% |
| | Total | 31 | 46 | 77 | Performance |
| | | | | | Agreement |
| | | | | | 100.0% |

### Example 5

### Testing of the Samples of Common Respiratory Viruses and Bacteria Using LAIV rRT-PCR Assays

The testing of the samples of common respiratory viruses and bacteria using LAIV rRT-PCR assays was performed. The data summarized in Table 9 indicated that both LAIV-A and LAIV-B assays showed no cross reactivity with common respiratory viruses and bacteria

TABLE 9
Specificity testing of LAIV rRT-PCR assays (LAIV-A and LAIV-B columns) using the
samples of common respiratory viruses and bacteria. The results of testing of the same
samples using CDC rRT-PCR Influenza Virus Panel (InfA and InfB columns) are shown for
comparison.

| Respiratory Pathogen | Strain | Titer | InfA | InfB | LAIV-A | LAIV-B |
|---|---|---|---|---|---|---|
| Viruses | | | | | | |
| Enterovirus | Echo 6 | 6.9^{a} | — | — | — | — |
| Human Adenovirus, type 1 | Ad. 71 | 9.2^{a} | — | — | — | — |
| Human Adenovirus, type 7a | S-1058 | 7.1^{a} | — | — | — | — |
| Human Coronavirus | OC43 | 50.4^{b} | — | — | — | — |
| Human Coronavirus | 229E | 31.6^{b} | — | — | — | — |
| Human Rhinovirus A | 1A | 5.8^{a} | — | — | — | — |
| Human Parainfluenza 1 virus | | 3.0^{b} | — | — | — | — |
| Human Parainfluenza 2 virus | Greer | 3.1^{a} | — | — | — | — |
| Human Parainfluenza 3 virus | C-243 | 7.9^{a} | — | — | — | — |
| Respiratory Syncytial Virus | CH93-18b | 6.8^{a} | — | — | — | — |
| (RSV) | | | | | | |
| Herpes Simplex Virus | KOS | 8.4^{a} | — | — | — | — |
| Varicella-zoster Virus | AV92-3 | 4.4^{a} | — | — | — | — |
| Epstein Barr Virus | B95-8 | 1.7^{b} | — | — | — | — |
| Measles Virus | Edmonston | 5.2^{a} | — | — | — | — |
| Mumps | Enders | 7.2^{a} | — | — | — | — |
| Cytomegalovirus | AD-169 | 6.9^{a} | — | — | — | — |
| Bacteria | | | | | | |
| Bordetella pertussis | A639 | 8.3^{d} | — | — | — | — |
| Candida albicans (yeast) | 2001-21-196 | 8.8^{d} | — | — | — | — |
| Chlamydia pneumoniae | TW183 | 40^{c} | — | — | — | — |
| Corynebacterium diphtheriae | — | 10^{d} | — | — | — | — |
| Escherichia coli | K12 | 9.6^{d} | — | — | — | — |
| Streptococcus pyogenes | 7790-06 | 7.5^{d} | — | — | — | — |
| Haemophilus influenzae | M15709 | 6.4^{d} | — | — | — | — |
| Lactobacillus plantarum | — | 8.8^{d} | — | — | — | — |
| Legionella pneumophila | — | 10.3^{d} | — | — | — | — |
| Moraxella catarrhalis | M15757 | 9.5^{d} | — | — | — | — |
| Mycobacterium tuberculosis | H37Rv | 95^{b} | — | — | — | — |
| Mycoplasma pneumoniae | M129 | 7.7^{d} | — | — | — | — |
| Neisseria elongata | — | 8.6^{d} | — | — | — | — |
| Neisseria meningitidis | M2578 | 7.9^{d} | — | — | — | — |
| Pseudomonas aeruginosa | — | 10.5^{d} | — | — | — | — |
| Staphylococcus aureus | — | 10.7^{d} | — | — | — | — |
| Staphylococcus epidermidis | — | 10.5^{d} | — | — | — | — |
| Streptococcus pneumoniae | 249-06 | 6.6^{d} | — | — | — | — |
| Streptococcus salivarius | SS1672 | 8.4^{d} | — | — | — | — |

^{a}Data represent TCID₅₀/ml;
^{b}Data represent ng/ml;
^{c}Data represent IFU/ml;
^{d}Data represent CFU/ml

### Example 4

### Determination of LOD of LAIV rRT-PCR Assays

LOD of LAIV rRT-PCR assays was determined using LAIV-A and LAIV-B virus strains. For comparison, LODs of the same virus strains were also determined using CDC rRT-PCR Influenza Virus Panel. The results are summarized in Table 10.

TABLE 10
LODs CDC rRT-PCR Influenza Virus Panel (InfA and InfB
columns) and LAIV assays (LAIV-A and LAIV-B columns).
(EID₅₀/ml or TCID₅₀/ml)

| LAIV-A virus | InfA | LAIV-A |
|---|---|---|
| A/Leningrad/134/17/1957_ca | 10^{2.56} | 10^{2.56} |
| LAIV_A virus (A/Wisconsin/67/2005)* | 10^{1.4} | 10^{1.4} |
| LAIV-B virus | InfB | LAIV-B |
| B/USSR/60/1969_ca | 10^{1.3} | 10^{1.3} |
| LAIV_B virus (B/New Mexico/07/2011)* | 10^{2.1} | 10^{2.1} |

*LAIV viruses (confirmed by sequence analysis)

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention as defined in the following claims.

1. A probe comprising an oligonucleotide comprising a sequence at least 90% identical to SEQ ID NO:3 or SEQ ID NO:6, the oligonucleotide linked to at least one of a fluorophore moiety and a fluorescence quencher moiety.

2-5. (canceled)

6. The probe of claim 1, wherein the fluorophore moiety comprises a fluorescein moiety.

7. (canceled)

8. The probe of claim 1, wherein the fluorescence quencher moiety is a BHQ quencher.

9-14. (canceled)

15. A kit for detecting a Live Attenuated Influenza Vaccine (LAIV) virus strain in a sample, comprising at least one probe of claim 1 and other reagents for performing a real time reverse transcriptase polymerase chain reaction (rRT-PCR) assay.

16. The kit of claim 15, wherein the other reagents comprise at least one primer comprising a sequence at least 90% identical to a sequence selected from the group consisting of SEQ ID NO:1, SEQ ID NO:2, SEQ ID NO:4 and SEQ ID NO:5.

17. The kit of claim 15, wherein the kit comprises
a probe comprising the sequence at least 90% identical to SEQ ID NO:3
and at least one of a first primer comprising a sequence at least 90% identical to SEQ ID NO: 1 and a second primer comprising a sequence at least 90% identical to SEQ ID NO:2.

18. The kit of claim 15, wherein the kit comprises
a probe comprising the sequence at least 90% identical to SEQ ID NO:6
and at least one primer of a first primer comprising a sequence at least 90% identical to a sequence SEQ ID NO:4 and a second primer comprising a sequence at least 90% identical to a sequence SEQ ID NO:5.

19. (canceled)

20. A kit for amplifying a region of a gene of a LAIV virus strain in a sample, comprising:
one or both of a first primer and a second primer for amplifying a region of PB1 gene of LAIV-A virus strain, the first primer comprising an oligonucleotide of a sequence at least 90% identical to SEQ ID NO: 1 and the second primer comprising an oligonucleotide of a sequence at least 90% identical to SEQ ID NO:2,
and/or one or both of a third primer and a fourth primer for amplifying a region of PA gene of LAIV-B virus strain, the third primer comprising an oligonucleotide of a sequence at least 90% identical to SEQ ID NO:4 and the fourth primer comprising an oligonucleotide of a sequence at least 90% identical to SEQ ID NO:5,
and one or more other reagents for performing a polymerase chain reaction (PCR).

21-25. (canceled)

26. The kit of claim 20, wherein the other reagents comprise at least one of
a first probe, comprising an oligonucleotide comprising a sequence at least 90% identical to SEQ ID NO:3, if the one or both primers for amplifying a region of PB1 gene of LAIV-A virus strain are preset in the kit, and
a second probe comprising an oligonucleotide comprising a sequence at least 90% identical SEQ ID NO:6, if one or both primers for amplifying a region of PA gene of LAIV-B virus strain are present in the kit.

27. The kit of claim 26, wherein the first probe and the second probe comprise at least one of a fluorophore moiety and a fluorescence quencher moiety.

28-29. (canceled)

30. A method of detecting a presence or absence of an influenza strain in a sample, wherein the influenza virus strain comprises a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising:
contacting the sample with reagents for performing a real time reverse transcriptase polymerase chain reaction (rRT-PCR) and
a probe of claim 1 specific for the region of LAIV-A PB1 gene (LAIV-A probe) and forward and reverse primers specific for the region of LAIV-A PB1 gene (LAIV-A primer), and/or
a probe of claim 1 specific for the region of LAIV-B PA gene (LAIV-B probe) and forward and reverse primers specific for the region of LAIV-B PA gene (LAIV-B primer),
performing rRT-PCR on the sample to generate a PCR cycle threshold;
comparing the PCR cycle threshold to a control value,
wherein if the PCR cycle threshold is below the control value, the LAIV virus strain is present in the sample, and wherein if the cycle threshold is above the control value, the LAIV virus strain is absent from the sample.

31. (canceled)

32. The method of claim 30, wherein the sample is contacted with LAIV-A probe and forward LAIV-A primer comprising a sequence at least 90% identical to SEQ ID NO:1.

33. The method of claim 30, wherein the sample is contacted with LAIV-A probe and a reverse LAIV-A primer comprising a sequence at least 90% identical to SEQ ID NO:2

34. The method of claim 30, wherein the sample is contacted with LAIV-B probe and forward LAIV-B primer comprising a sequence at least 90% identical to SEQ ID NO:4.

35. The method of claim 30, wherein the sample is contacted with LAIV-B probe and a reverse LAIV-B primer comprising a sequence at least 90% identical to SEQ ID NO:5.

36. (canceled)

37. The method of claim 30, wherein the comparing is performed by a computer.

38. A method of amplifying a region of a gene of LAIV virus strain in a sample, wherein the region is a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising:
contacting the sample with reagents for performing a polymerase chain reaction (PCR), the reagents comprising
at least one of a first primer and a second primer comprising an oligonucleotide of a sequence at least 90% identical to SEQ ID NO: 1 and a second primer comprising an oligonucleotide of a sequence at least 90% identical to SEQ ID NO:2,
and/or at least one of a third primer comprising an oligonucleotide of a sequence at least 90% identical to SEQ ID NO:4 and a fourth primer comprising an oligonucleotide of a sequence at least 90% identical to SEQ ID NO:5; and,
performing the PCR.

39-40. (canceled)

41. A method of detecting the LAIV virus strain in the sample, comprising:
performing the method of claim 38; and,
detecting one or more products of the amplification,
wherein the LAIV influenza strain is present in the sample if the one or more products of the amplification are detected corresponding to the region of LAIV-A PB1 gene or LAIV-B PA gene.

42-45. (canceled)

46. A method of determining if a patient is infected with a LAIV virus strain, wherein the LAIV virus strain comprises a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising:
contacting a sample derived from the patient with reagents for performing a real time reverse transcriptase polymerase chain reaction (rRT-PCR), the reagents comprising
a probe of claim 1 specific for the region of LAIV-A PB1 gene (LAIV-A probe) and forward and reverse primers specific for the region of LAIV-A PB1 gene (LAIV-A primer), and/or
a probe of claim 1 specific for the region of LAIV-B PA gene (LAIV-B probe) and forward and reverse primers specific for the region of LAIV-B PB-A gene (LAIV-B primer),
performing the rRT-PCR on the sample to generate a PCR cycle threshold; and,
comparing the PCR cycle threshold to a control value,
wherein if the cycle threshold is below the control value, the patient is infected with the LAIV virus strain, and wherein if the cycle threshold is above the control value, the patient is not infected with the LAIV virus strain.

47-52. (canceled)

53. A method of determining if a patient is infected with a LAIV virus strain, wherein the LAIV virus strain comprises a region of PB1 gene of influenza virus derived from attenuated cold-adapted influenza virus A/Ann Arbor/06/1960-ca or A/Leningrad/134/1957-ca (LAIV-A PB1 gene), or a region of PA gene of LAIV-B virus derived from attenuated cold-adapted influenza virus B/Ann Arbor/01/1966-ca or B/USSR/60/1969-ca (LAIV-B PA gene), the method comprising:
performing a polymerase chain reaction (PCR) on a sample derived from the patient using a kit of claim 20; and,
detecting one or more products of the PCR,
wherein the LAIV virus strain is present in the sample if the one or more products of the amplification are detected corresponding to a gene region derived from LAIV-A or LAIV-B.

54-55. (canceled)
